(19) United States
(12) Plant Patent Application Publication
(10) Pub. No.: US 2007/0136909 P1
(43) Pub. Date: Jun. 14, 2007

(54) Gaura plant named 'Gaudros'

(75) Inventor: Henricus G.W. Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/299,619

(22) Filed: Dec. 12, 2005

(51) Int. Cl. A01H 5/00

(52) U.S. Cl. PLT/263

(57) ABSTRACT

A new and distinct variety of Gaura plant, substantially as herein illustrated and described, characterized particularly as to novelty by a well-branched, compact habit, continuous flowering, pink flowers, green foliage and early to flower.

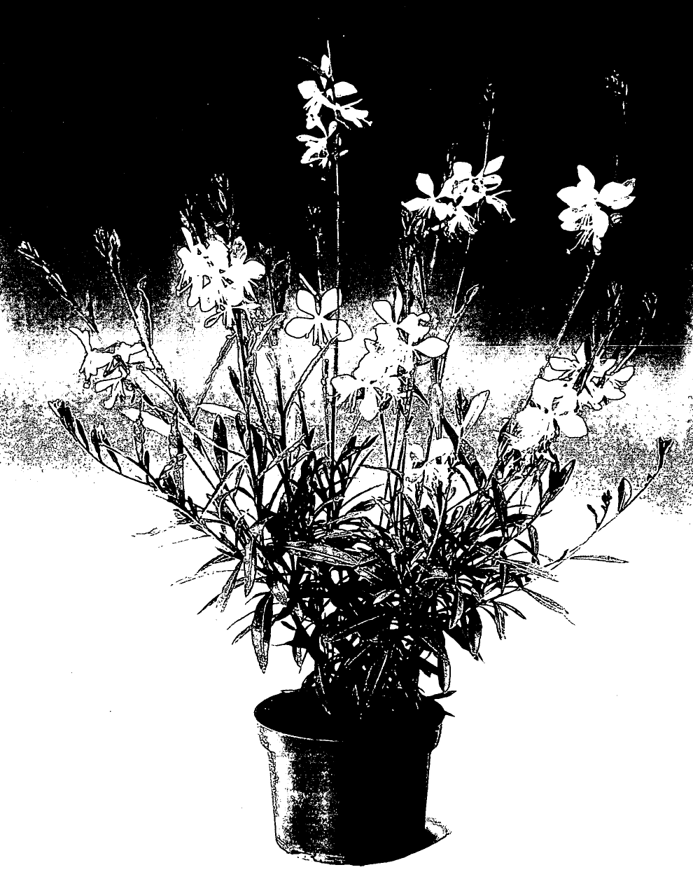

### LATIN NAME OF THE GENUS AND SPECIES OF THE PLANT CLAIMED

Gaura lindheimeri

### VARIETAL DENOMINATION

‘Gaudros’

### BACKGROUND OF THE NEW PLANT

The present invention comprises a new and distinct Gaura plant, botanically known as Gaura lindheimeri, and hereinafter referred to by the cultivar name ‘Gaudros.’

The new cultivar was developed by the inventor in a controlled breeding program during 2000, at Enkhuizen, The Netherlands. The new Gaura cultivar is characterized by a well-branched, compact habit, continuous flowering, green foliage and early to flower.

The female (seed) parent of ‘Gaudros’ was ‘B0012-1,’ an unpatented proprietary inbred line that is not known by any synonyms. The male (pollen) parent of ‘Gaudros’ was ‘C0013-1,’ also a proprietary inbred line that is not known by any synonyms. ‘Gaudros’ was selected in May 2001 as a single flowering plant from within the F2 progeny of the above stated cross and was initially designated ‘D0010-1.’

Asexual reproduction of the new cultivar at Enkhuizen, The Netherlands and Sarrians, France was carried out by the use of terminal stem cuttings, and has demonstrated that the new variety is stable and reproduces true to type in successive generations of asexual reproduction.

### SUMMARY OF THE INVENTION

It was found that the cultivar of the present invention:
a) Exhibits pink flowers;
b) Forms green foliage; and
c) Exhibits an upright and very well branched growth habit

### DESCRIPTION OF THE DRAWING

This new Gaura plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Gaura. The data which defines these characteristics were collected from asexual reproductions carried out in Enkhuizen, Netherlands. The plant history was taken on 16 weeks old plants blossomed under natural light in a greenhouse and grown in a 13 cm container.

Color readings were taken in the greenhouse under ambient light. Color references are primarily to the RHS Colour Chart of The Royal Horticultural Society of London.

TABLE 1
Differences between the new cultivar ‘Gaudros,’ its
parents and a similar cultivar

| | ‘Gaudros’ | ‘B0012-1’ | ‘C0013-1’ | ‘Siskyou Pink’ |
|---|---|---|---|---|
| Flower color | Pink | Rose | White | Light pink |
| Height | 50 cm | 80 cm | 60 cm | 60 cm |
| Branching | Very good | Good | Poor | Poor |

‘Siskyou pink’ is not patented.

The plant:
   Classification.—Botanical: Gaura — Species: Lindheimeri.
   Parentage.—Female parent: A seedling named ‘B0012-1’ is one of our seedlings from our B-generation of plants bred in 1999. — Pollen parent: A seedling named ‘C0013-1’ is one of our seedlings from our C-generation of plants bred in 2000.
   Growth habit.—Upright.
   Plant height.—50-70 cm.
   Spreading area of plant.—20-30 cm.
   Strength.—Very resistant to hot and cold weather.
   Branching habit.—Freely branching and lateral branching at every node.
   Blooming period.—From May until November.
   Propagation.—Vegerative stem cuttings.
   Sexuality.—Hermaphrodite.
   Time to initiate roots.—Approximately 12 days at temperatures of 22 degrees centigrade is needed to produce rooted cuttings.
   Root system.—Fibrous, branching, the color is N157C.
The stem:
   Diameter.—2-4 mm.
   Length.—50-70 cm.
   Shape.—Round.
   Anthocyan pigmentation.—Present, color 185A.
   Color of the stem.—138C.
   Length of internode.—20-40 mm.
   Pubescence.—Slightly pubescent.
The foliage:
   Phyllotaxis.—Alternate.
   Shape of blade.—Lanceolate to oblanceolate.
   Attachment.—Sessile.
   Texture.—Upper side: Slightly pubescent — Lower side: Slightly pubescent.
   Venation.—Pinnate.
   Leaf margin.—Entire to very slightly dentate.
   Leaf base.—Attenuate.
   Leaf apex.—Acute.
   Length.—20-45 mm.
   Width.—10-23 mm.
   Depth of incision.—Less than 1 mm.
   Color.—Upper side: 146A — Lower side: 146B.
   Color of venation.—Upper side: 143B — Lower side: 143C.
The bud:
   Pedicel length.—3-6 mm.
   Pedicel diameter.—1 mm.
   Pedicel color.—181B.
   Pedicel surface.—Slightly hairy.
   Stipule length.—1-3 mm.
   Stipule width.—1-2 mm.
   Stipule shape.—Lanceolate.
   Stipule color.—Upper side: 146C — Lower side: 146C.
   Bud size.—Diameter: 2 mm — Length: 6-8 mm.
   Bud shape.—Tubular.
   Bud colour.—182D.
   Bud surface.—Pubescent.
   Sepals.—Color (Upper side): 182D — Color (Lower side): 182D — Form: Upright — Surface: Pubescent — Number: 4, fused at tip — Length: 7-11 mm — Width: 1.5-2.5 mm — Shape: Tubular — Apex: Acute — Base: Subcordate — Margin: Entire.
The flower:
   Type of inflorescence.—Spicate raceme.
   Flower shape.—Single, cruciform.
   Flower color.—Upper side: 68B — Lower side: 68A.
   Flower size.—Height: 14-16 mm — Width: 18-20 mm.
   Petals, number.—4, non-overlapping.
   Petal shape.—Oblanceolate.
   Petal margin.—Entire.
   Petal apex.—Acute to obtuse.
   Petal base.—Attenuate.
   Petal length.—7-9 mm.
   Petal width—7-9 mm.
   Petal texture.—Smooth.
   No. of flowers per inflorescence.—40-60.
   Length of inflorescence (end).—25-60 cm.
   Fragrance.—No fragrance.
   Bloom time of one inflorescence.—New florets continue to open in one inflorescence over a period of 25 days.
   Lastingness of one flower.—34 days.
   Number of flowers open at one time in one inflorescence.—1-2.
The reproductive organs:
   Androecium.—Stamens quantity: 8 Stamen length: 12 mm Stamen colour: N155A Anther shape: Elongated Anther length: 0.8 mm Anther width: 0.2 mm Anther color: 9D Pollen amount: Plentiful pollen Pollen color: 9D.
   Gynoecium.—Pistils quantity: 1 Pistil length: 12 mm Stigma color: 4D Stigma shape: Lobed Number of lobes: 4 Style color: N155A Ovary color: 141D Ovary position: Inferior Ovary shape: Elongated Ovary dimensions: 0.2 mm in width and 0.5 mm in length.
Seeds: One seed is formed per flower
   Seed size.—Length: 3 mm —Width: 1 mm.
   1000 seeds weight.—4.5 grams.
   Seed color.—185B.
   Seed texture.—Smooth.
Disease resistance: Resistance to pathogens has not been observed.
Hardiness zone: ‘Gaudros’ is hardy in zones eight (8) and above.

What is claimed is:

1. A new and distinct cultivar of Gaura plant named ‘Gaudros,’ as substantially illustrated and described herein.
